(19) United States
(12) Patent Application Publication
(10) Pub. No.: US 2004/0131672 A1
(43) Pub. Date: Jul. 8, 2004

(54) Direct compression pharmaceutical composition containing a pharmaceutically active ingredient with poor flowing properties

(76) Inventors: Nilobon Podhipleux, Weston, FL (US); Unchalee Kositprapa, Davie, FL (US); Avinash Nangia, Weston, FL (US)

Correspondence Address: Martin P. Endres, Esq., HEDMAN & COSTIGAN, P. C., 1185 Avenue of the Americas, New York, NY, 10036, US

(21) Appl. No.: 10/337,444

(22) Filed: Jan. 7, 2003

(51) Int. Cl. A61K 9/20

(52) U.S. Cl. 424/465

(57) ABSTRACT

The present invention relates to a directly compressible pharmaceutical composition that contains greater than 15 weight percent of a poorly flowing drug powder, greater than 2 weight percent of a super disintegrant and greater the 1 weight percent of a glidant.

### FIELD OF THE INVENTION

The present invention relates to the field of oral dosage forms and in particular to a composition that can be used to prepare oral pharmaceutical dosage forms by direct compression. More specifically, the present invention relates to a composition that can be directly compressed into oral dosage forms which contain drugs or pharmaceutically active ingredients such as sertraline hydrochloride which exhibit poor flow properties. The tablets prepared from the composition of the present invention can be further coated with a seal coat, taste masking coat, color coat, enteric coat, controlled release coat or any combination of the aforementioned coatings.

### BACKGROUND OF THE INVENTION

Direct compression is a well known technique in the pharmaceutical industry for forming tablets or tablet cores. The technique is described in Remington's Pharmaceutical Sciences, 18^{th} edition, pages 1633-58 and in The Theory and Practice of Industrial Pharmacy, by Lachman et al., 2^{nd} edition, pages 321-358 which are incorporated herein by reference. In general, direct compression involves forming tablets by applying or compressing powder material without modifying the physical nature of the powder material to be compressed. The powder materials used in direct compression should possess good adhesive, cohesive and flow properties. The adhesive and flow properties can be adjusted by changing the composition of the powder to be compressed (i.e. adding excipients such as binders or glidants or by varying the amounts of the ingredients) or by preprocessing powders before compressing them such as subjecting the powders to a wet granulation or slugging step.

Varying the ingredients or preprocessing does not guarantee that the powders can be successfully compressed. For example, there are many drugs that are “fluffy” in nature or that exhibit relatively low bulk and tap density, making it difficult to formulate a large amount of these drugs into tablets with a uniformity of weight and hardness when direct compression is used. Also many drugs have undesirable characteristics such as stickiness to punches and dies. This problem is amplified when high speed tablet presses are used. Further, high levels of lubricants and/or glidants can result in non-uniform tablets or tablets that exhibit capping, lamination or chipping. Similarly, adding excipients, increases the size of the tablets or cores making the final dosage formulation difficult to swallow as well as affecting the release of the drug from the dosage formulation. Preprocessing adds additional steps to a process thereby increasing processing time and the chances of error. The literature suggests that three major factors influence the flowability of powders: 1) particle properties; 2) the environmental conditions; and 3) the testing methods. Particle properties include the particle size, shape and particle size distribution. The spherical and oblong shaped particles flow easily while the sharp edged particles flow less readily. The flow becomes poorer with irregularly shaped particles and flow becomes adversely affected by the formation of bridges that tend to occur primarily with plate shaped and fibrous type particles. The cohesive powder poor flow is due to the large surface area available for interparticular friction resulting in the development of electrostatic charges. Other properties that tend to affect the flow are the particle density and particle elastic and plastic deformation properties. The environmental conditions that may affect flow are the humidity and moisture content and if the powder adsorbs gases and other impurities from the surrounding atmosphere.

Some direct compressible formulations for medications such as sertraline have been developed. Formulations such as the ones found in U.S. Pat. Nos. 5,853,758, 4,536,518 and 5,876,752 which are incorporated herein by reference utilize a meltable binder or polymerized membrane, compressed with excipients and a pharmaceutically active agent to improve tablet strength.

It is therefore an objective of the present invention to provide a directly compressible composition that employs a high amount of a poor flowing drug and a high amount of lubricant and/or glidant which can be formed into a tablet or core that does not exhibit capping, lamination or chipping while keeping the tablet or core small.

It is also an object of the present invention to provide a directly compressible composition that employs a super disintegrant to allow release of the poor flowing drug from the core.

It is a further object of the present invention to provide a directly compressible composition that contains a poor flowing drug and a super disintegrant that can be easily formed into a tablet or core and subsequent coated with a polymeric material to mask an unpleasant taste of the drug, provide for controlled release of the drug or impart an esthetic appearance to the tablet or core.

It is another object of the present invention to provide a directly compressible composition that can be formed into a core tablet or core that employs a high amount of a poor flowing drug, a high amount of lubricant and/or glidant and a super disintegrant.

### SUMMARY OF THE INVENTION

The foregoing objectives are obtained by a composition comprising at least 15% by weight of a poor flowing drug, at least 2% by weight of a super disintegrant, and at least 1% by weight of a glidant. In a preferred embodiment of the present invention, the composition should comprise at least 20%, most preferably at least 25%, by weight of a poor flowing drug, at least 3%, most preferably at least ⁴%, by weight of a super disintegrant, and at least 1.5, most preferably at least 2%, by weight of a glidant. The composition may also contain other conventional excipients such as fillers, stablizers and lubricants. The above mentioned ingredients are mixed prior to compression using standard techniques known in the industry. The mixture is then placed into a hopper or feed frame that dispenses the mixture without impedance into the die cavity of a tablet press for compression into tablets or cores.

As used in this application, the term “poor flowing drug” refers to a drug or drug mixture that contains 25% or more by weight of drug, without lubricant or glidant and that will not flow or create a “rathole” when fed through a hopper of a conventional press. More specifically, a poor flowing drug is a drug that exhibits an angle of repose greater than 60°. An angle of repose is the maximum angle at which a pile of unconsolidated material may remain stable.

### DETAILED DESCRIPTION OF THE INVENTION

The pharmaceutical composition of the invention comprises a pharmaceutically active compound, e.g. drug or drugs that exhibits poor flow properties. Examples of poor flowing drugs include but are not limited to irbesartan, hydrochlorothiazide, clodronate, antacid drugs such as aluminum hydroxide and magnesium carbonate, antidepressants such as sertraline and paroxetine, HMG-CoA reductase inhibitors such as lovastatin, pravastatin, simvastatin, mevastatin and atorvastatin and pharmaceutically acceptable salts, isomers and metabolites of the foregoing. The amount of drug in the composition is about 15 to about 90 weight percent, preferably, about 20 to about 75 weight percent and most preferably about 25 to about 50 weight percent.

The pharmaceutical composition also comprises a super disintegrant. A super disintegrant is an excipient that swells four to forty fold in less than 30 seconds when placed in an aqueous medium. Some examples of super disintegrants are Veegum HV, methylcellulose, agar, bentonite, cellulose and wood products, natural sponge, pectins, cation exchange resins, alginic acid, guar gum, citrus pulp, starch, and carboxymethylcellulose. The preferred super disintegrants are crosscarmellose sodium, crospovidone (Polyplasdone® XL, Kollidon® CL), sodium starch glycolate (Primojel®, Explotab®) or mixtures of the foregoing. The super disintegrant comprises about 2 to about 15 weight percent of the composition, preferably about 3 to about 10 weight percent and most preferably about 4 to about 8 weight percent of the composition.

The composition also comprises a glidant. A glidant is an excipient that improves the flow characteristics of the compressible powder. Two of the most common glidants are colloidal silicon dioxide (Cab-O-Sil) and Quso (also known as Phila Quartz). The amount of glidant that is commercially used ranges from about 0.1 to about 0.5 weight percent. The amount of glidant used in the present invention exceeds the amount that is commercially used and ranges generally from about 1 to about 10 percent of the composition, preferably about 1.5 to about 8 percent and most preferably about 2 to about 5 percent.

The composition of the invention further comprise pharmaceutically acceptable fillers, such as compressible sugar, lactose, glucose, sucrose, mannitol, and binders, such as polyvinylpyrrolidone, microcrystalline cellulose, methylcellulose, hydroxypropyl cellulose, hydroxymethyl cellulose, hydroxypropyl methylcellulose and combinations of the foregoing. The amount of filler is about 25 to about 75 percent of the composition and preferably about 35 to about 65 percent of the composition. In a preferred embodiment the filler is a mixture of water soluble and water insoluble materials such as a sugar and a cellulosic material. If the combination of water soluble and water insoluble fillers is used the ratio of water soluble to water insoluble filler is about 1:4 to about 4:1, preferably about 1:2 to about 2:1 and most preferably about 1:1.

Lubricants may also be added to the composition. A lubricant is a material that reduces or prevent the adhesion of the composition to a die or punch and thereby allows the compressed tablet or core to be easily removed for the die or punch. Commonly used lubricants are talc, magnesium stearate, calcium stearate, stearic acid, hydrogenated vegatable oils and polyethylene glycols. The amount of lubricant employed in the composition should be about 0.5 to about 5 percent, preferably about 1 to about 3 percent.

Depending upon the drug employed in the composition a stabilizing or buffering agent may be added to the composition. Some common stabilizers and/or buffering agents are phosphates, silicates, carbonates, amino acids (lysine, arginine, ornithine, histidine), fatty acids, organic acid (citric acid, fumaric acid, tartaric acid, caproic acid), organic buffering compounds (tromethamine, N-amino sugars), ammonium salts and aluminium salts (aluminium hydroxide). If a stabilizer or buffering agent is employed in the formulation, it comprises about 2 to about 30 percent, preferably about 5 to 25 percent and most preferably about 10 to about 20 percent of the composition.

Once the tablet or core is prepared it can be coated with an esthetic or seal coating, taste mask coating, enteric coating, or controlled release coating. The composition and methods for applying these coating are well known in the art. Examples of esthetic or seal coatings are described in U.S. Pat. Nos. 5,922,352 and 6,210,716 and are incorporated herein by reference. Typical enteric coatings are described in U.S. Pat. Nos. 6,013,281; 6,077,541; and 6,174,548 and are incorporated herein by reference. Typical controlled release coatings are describes in U.S. Pat. Nos. 6,210,716; 6,270,805; 5,439,689; 4,890,240; 6,099,859; 6,099,862 and 4,61,008 and are incorporated herein by reference.

In a preferred embodiment, the compressed tablet or core is coated with a water soluble polymer or polymer that is soluble in the gastric fluid such as Eudragit E with or without a pigment. The coating is applied by forming a solution of the respective coating material in an organic solvent such as acetone and isopropyl alcohol and employing any of the application techniques known to those skilled in the art, such as fluid bed coating or pan coating. Core tablets can also be coated with water soluble polymers by press-fit technology.

The following example illustrates the present invention and is not intended to limit the scope of the present invention.

### EXAMPLE 1

### Preparation of A Directly Compressible Composition

A 1.7 kg batch of directly compressible composition in accordance with the present invention is prepared having the following ingredients:

| | | |
|---|---|---|
| Sertraline HCl, USP | 494.19 g | (29.07%) |
| Microcrystalline Cellulose, NF | 389.98 g | (22.94%) |
| Dicalcium Phosphate Dihydrate Powder, USP | 259.93 g | (15.29%) |
| Lactose Monohydrate, NF | 397.46 g | (23.38%) |
| Colloidal Silicon Dioxide, NF | 41.99 g | (2.47%) |
| Sodium Starch Glycolate, NF | 99.28 g | (5.84%) |
| Magnesium Stearate, NF | 17.17 g | (1.01%) |

The composition was prepared by adding 494.19 g of Sertraline HCl, 389.98 g of microcrystalline cellulose (Avicel PH 102), 259.93 g of dicalcium phosphate dihydrate USP/FCC powder, 397.46 g of lactose monohydrate, NF, (modified-spray dried), 41.99 g of colloidal silicon dioxide, NF and 99.28 g of sodium starch glycolate, NF (EXPLOTAB pH 5.5-7.5) to a blender and mixing for approximately 20 minutes at a speed of about 23 rpm. After blending, the mixture is passed through a Comil with a #1143 size stainless steel screen, 0.175″ spacer.

After the mixture passes through the Comil, it is blended again for an additional 20 minutes. Once the second blending is completed, 17.17 g of magnesium stearate that has been previously screened through a 30 mesh stainless steel screen is added to the mixture in the blender and mixed again for approximately five minutes.

The mixture is then compressed into tablets using the following equipment:

| | |
|---|---|
| Tooling size: | 0.2500″ × 0.4550″ |
| Shape | Capsule-shaped |
| Upper Punch | Bisect |
| Lower Punch | Plain |

The average weight of the resulting tablets is 385.0 mg, with a hardness between 8-12 kp.

### EXAMPLE 2

The tablets prepared in Example 1 are coated with a seal/taste masking coat having the following composition:

| | |
|---|---|
| Eudragit E-100 | 12.5% |
| Isopropyl Alcohol (99%) | 52.5% |
| Acetone, NF | 35% |

The above seal/taste masking solution is applied to the tablets using an O'Hara Perforated Coating Pan under the following conditions:

| | |
|---|---|
| Exhaust Temperature | 30° C. |
| Air Volume | 220-240 SCFM |
| Atomization Pressure | 30 psi |
| Pan Speed | 4-8 rpm |
| Spray Rate | 1-25 ml/min |

The tablets are coated until approximately 10 mg of the Eudragit E-100 is applied.

### EXAMPLE 3

The seal/taste masked coated tablets prepared in Example 2 are color coated with the following suspension:

| | |
|---|---|
| OPADRY ® Yellow (YS-1-6318) | 10% |
| Purified Water, USP | 90% |

The above color coating suspension is applied to the tablets using an O'Hara Perforated Coating Pan under the following conditions:

| | |
|---|---|
| Exhaust Temperature | 40° C. |
| Air Volume | 220-280 SCFM |
| Atomization Pressure | 30 psi |
| Pan Speed | 4-8 rpm |
| Spray Rate | 10-20 ml/min |

The tablets are coated until approximately 12.22 mg of the OPADRY® Yellow is applied. The color coated tablets are then polished with candelilla wax.

The tablet prepared in this example were analyzed in human patients using standard techniques known in the art. The testing involved two panels of randomly selected patients that received either the tablet formulation prepared in this Example or ZOLOFT® a commercially available tablet form of sertraline (Lot #9J097E) in an open, randomized single dose study. Blood samples were collected over a 120 hour period and analyzed for sertraline concentrations with a LC/MS/MS method. The results of this in vivo testing is provided below:

FED (N = 6)

| | Test Mean | Ref. Mean | G-Mean Ratio |
|---|---|---|---|
| Cₘₐₓ (ng/ml) | 41.52 | 42.35 | 0.982 |
| AUC₀₋ₜ (ng · hr/ml) | 988.78 | 975.48 | 1.010 |
| AUC_{o-inf} (ng · hr/ml) | 1020.27 | 1014.37 | 1.004 |
| Tₘₐₓ (hr) | 6.00 | 5.67 | 1.070 |

FASTING (N = 8)

| | Test Mean | Ref. Mean | G-Mean Ratio |
|---|---|---|---|
| Cₘₐₓ (ng/ml) | 31.76 | 30.25 | 1.037 |
| AUC₀₋ₜ (ng · hr/ml) | 890.79 | 864.89 | 1.018 |
| AUC_{o-inf} (ng · hr/ml) | 937.69 | 944.06 | 0.978 |
| Tₘₐₓ (hr) | 7.00 | 7.25 | 0.965 |

### EXAMPLE 4

The tablets prepared in Example 1 can be coated with a combined color and taste masking coat having the following composition:

| | |
|---|---|
| Eudragit E-100 | 3.125% |
| Chroma-Teric (DEB-5123-YE) | 9.375% |
| Isopropyl Alcohol (99%) | 52.5% |
| Acetone, NF | 35.0% |

The above color/taste masking suspension can be applied to the tablets using an O'Hara Perforated Coating Pan under the following conditions:

| | |
|---|---|
| Exhaust Temperature | 30° C. |
| Air Volume | 220-240 SCFM |
| Atomization Pressure | 30 psi |
| Pan Speed | 4-8 rpm |
| Spray Rate | 1-25 ml/min |

The tablets should be coated until approximately 4 mg of the Eudragit E-100 and 12 mg of Chroma-Teric is applied. The color/taste masked tablet is polished with candelilla wax.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

We claim:

1. A pharmaceutical composition comprising:
a) at least 15 weight percent of a poorly flowing drug powder;
b) at least 2 weight percent of a super disintegrant;
c) at least 1 weight percent of a glidant;
d) about 25 to about 75 weight percent of a filler;
e) 0 to about 30 weight percent of a stabilizer; and
f) 0.5 to 5 weight percent of a lubricant wherein the composition is directly compressible into a tablet or tablet core.

2. The composition as defined in claim 1 wherein the drug comprises at least 20 weight percent of the composition.

3. The composition as defined in claim 2 wherein the drug comprises at least 25 weight percent of the composition.

4. The composition as defined in claim 1 wherein the super disintegrant comprises at least 3 weight percent of the composition.

5. The composition as defined in claim 4 wherein the super disintegrant comprises at least 4 weight percent of the composition.

6. The composition as defined in claim 1 wherein the glidant comprises at least 1.5 weight percent of the composition.

7. The composition as defined in claim 6 wherein the glidant comprises at least 2 weight percent of the composition.

8. The composition as defined in claim 1 wherein the filler comprises about 35 to about 65 weight percent of the composition.

9. The composition as defined in claim 1 wherein the lubricant comprises about 1 to about 3 weight percent of the composition.

10. The composition as defined in claim 1 wherein the stabilizer comprises about 2 to about 30 weight percent of the composition.

11. The composition as defined in claim 10 wherein the stabilizer comprises about 5 to about 25 weight percent of the composition.

12. The composition as defined in claim 11 wherein the stabilizer comprises about 10 to about 20 weight percent of the composition.

13. The composition as defined in claim 1 wherein the filler is a mixture of water soluble fillers and water insoluble fillers.

14. The composition as defined in claim 13 wherein the ratio of water soluble filler to water insoluble filler is about 1:4 to about 4:1.

15. The composition as defined in claim 14 wherein the ratio of water soluble filler to water insoluble filler is about 1:2 to about 2:1.

16. The composition as defined in claim 15 wherein the ratio of water soluble filler to water insoluble filler is about 1:1.

17. The composition as defined in claim 1 wherein the drug is sertraline or its pharmaceutically acceptable salt, isomer or metabolite, the glidant is colloidal silicon dioxide and the super disintegrant is sodium starch glycolate.

18. The composition of claim 17 wherein the stabilizer is dicalcium phosphate dihydrate.

19. The composition as defined in claim 1 further comprising an esthetic coat, a seal coat, a taste masking coat or a controlled release coating applied to the tablet or tablet core.

20. A pharmaceutical composition consisting essentially of:
a) 15-90 weight percent of sertraline, its pharmaceutically acceptable salt, isomer or metabolite;
b) 2-15 weight percent of a super disintegrant;
c) 1-10 weight percent of a glidant;
d) 25-75 weight percent of a filler;
e) 2-30 weight percent of a stabilizer; and
f) 0.5 to 5 weight percent of a lubricant wherein the composition is directly compressible into a tablet or tablet core; and
g) the tablet or tablet core is optionally coated with an esthetic coat, a seal coat, a taste masking coat or a controlled release coating.

21. The composition as defined in claim 20 wherein the glidant is colloidal silicon dioxide and the super distintegrant is sodium starch glycolate.

22. The composition as defined in claim 20 wherein the filler is a mixture of water soluble fillers and water insoluble fillers.

23. The composition as defined in claim 22 wherein the ratio of water soluble filler to water insoluble filler is about 1:4 to about 4:1.

24. The composition as defined in claim 23 wherein the ratio of water soluble filler to water insoluble filler is about 1:2 to about 2:1.

25. The composition as defined in claim 24 wherein the ratio of water soluble filler to water insoluble filler is about 1:1.

26. A pharmaceutical composition consisting essentially of:
a) 20-75 weight percent of sertraline, its pharmaceutically acceptable salt, isomer or metabolite;
b) 3-10 weight percent of a super disintegrant;
c) 1.5-8 weight percent of a glidant;
d) 25-75 weight percent of a filler;
e) 5-25 weight percent of a stabilizer;
f) 0.5 to 5 weight percent of a lubricant wherein the composition is directly compressible into a tablet or tablet core; and
g) optionally an esthetic coat, a seal coat, a taste masking coat or a controlled release coating applied to the tablet or tablet core.

27. The composition as defined in claim 26 wherein the filler is a mixture of water soluble fillers and water insoluble fillers.

28. The composition as defined in claim 26 wherein the ratio of water soluble filler to water insoluble filler is about 1:4 to about 4:1.

29. The composition as defined in claim 28 wherein the ratio of water soluble filler to water insoluble filler is about 1:2 to about 2:1.

30. The composition as defined in claim 29 wherein the ratio of water soluble filler to water insoluble filler is about 1:1.

31. A pharmaceutical composition consisting essentially of:
a) 20-50 weight percent of sertraline, its pharmaceutically acceptable salt, isomer or metabolite;
b) 4-8 weight percent of a super disintegrant;
c) 2-5 weight percent of a glidant;
d) 35-65 weight percent of a filler;
e) 10-20 weight percent of a stabilizer;
f) 1-3 weight percent of a lubricant wherein the composition is directly compressible into a tablet or tablet core; and
g) optionally an esthetic coat, a seal coat, a taste masking coat or a controlled release coating applied to the tablet or tablet core.

32. The composition as defined in claim 31 wherein the filler is a mixture of water soluble fillers and water insoluble fillers.

33. The composition as defined in claim 31 wherein the ratio of water soluble filler to water insoluble filler is about 1:4 to about 4:1.

34. The composition as defined in claim 33 wherein the ratio of water soluble filler to water insoluble filler is about 1:2 to about 2:1.

35. The composition as defined in claim 34 wherein the ratio of water soluble filler to water insoluble filler is about 1:1.
